(19) United States
(12) Patent Application Publication
(10) Pub. No.: US 2025/0025335 A1
(43) Pub. Date: Jan. 23, 2025

(54) METHOD AND DEVICE FOR IRRIGATION INTO THE LACRIMAL PUNCTA

(71) Applicant: VISANT MEDICAL, INC., Menlo Park, CA (US)

(72) Inventor: Cary REICH, Santa Barbara, CA (US)

(21) Appl. No.: 18/574,877

(22) PCT Filed: Jun. 29, 2021
(86) PCT No.: PCT/US21/39677

§ 371 Date: Dec. 28, 2023

(51) Int. Cl. A61F 9/007

(52) U.S. Cl. CPC A61F 9/007

(57) ABSTRACT

A lacrimal cannula includes a hollow tubular body having a dispensing end, a loading end, and an injection lumen running from the loading end to the dispensing end. The dispensing end terminates in a distal tip configured to engage and seal against a punctal os in a patient's eye while being inhibited from entering a horizontal portion of the lacrimal canaliculus. The distal tip has an outer diameter selected to allow limited entry into an upper region of a vertical portion of the lacrimal canaliculus. A region of the hollow tubular body proximal of distal tip is sufficiently large to block advancement of the distal tip beyond a short distance into the vertical portion of the lacrimal canaliculus, typically no more than 2 mm.

### BACKGROUND OF THE INVENTION

1. Field of the Invention. The present invention relates generally to medical devices and methods and more particularly to a device for inserting materials through the lacrimal puncta and into the lacrimal canalicula, and lacrimal sac.

Lacrimal cannulas are known and have been used to access the lacrimal canaliculi (ducts) through the puncta of the eye. Fluid is delivered from the cannula into the lacrimal canaliculi to clear the ductwork of obstructions, increase drainage of the tears, and in some cases to flush lacrimal implants. Lacrimal cannula, in general, may be constructed similarly to intravenous needles and catheters, with early examples illustrated in U.S. Pat. Nos. 950,822 and 3,388,703 respectively.

In the past, lacrimal cannulas have typically had a uniform outer diameter, for example in a range from 27 gauge to 21 gauge or had a tapered exterior surface, as described in U.S. Pat. No. 5,593,393. Such a single-diameter cannula typically has a larger diameter (smaller gauge number) making insertion into the puncta of the eye difficult. Before introducing such large, single diameter cannula, the puncta typically requires extensive dilation with a punctal dilating instrument, making punctal access a two-step process. In some instances, lacrimal duct probing may also be required, making ductal access a three-step procedure. Smaller diameter (larger gauge number) cannulas are more readily inserted but provide an inferior seal which can allow fluid backflush once irrigation initiated.

Previous punctal insertion devices also lack the ability to allow a user to estimate penetration depth into the lacrimal canalicula (which is only 10 mm in its entirety). Lack of such feedback can result in “over insertion” of the cannula (to a penetration depth greater that need and beyond the avail punctal length) potentially bruising internal tissue at the end of the canaliculi.

FIG. 1 illustrates the anatomy of the lacrimal system of the human eye 10 including a lacrimal gland 12, a lower lacrimal punctal os 14a, an upper lacrimal punctal os 14b, a lacrimal sac 16, and a lower canaliculus 18. The lacrimal canaliculus 18 includes of an approximately 2 mm vertical section or portion that descends from the punctal os14a and an 8 mm horizontal section or portion extending to the lacrimal sac 16. Tears 22 flow from the lacrimal gland 12 over the eye onto a top surface 28 of a lower eye lid to a lower lacrimal punctal os 14a, through the lacrimal canaliculus 18, and to the lacrimal sac 16.

FIGS. 2A and 2B illustrate a typical prior art lacrimal injector system including a dilator 41 used to expand the lower lacrimal punctal os 14a to allow insertion of a lacrimal cannula 42, typically having an outer diameter from 0.3 mm to 1 mm, attached to a syringe 40 having finger grips 47 containing a gel or other material 44 to be inserted into the lacrimal canaliculus 18 through the lower lacrimal punctal os 14a.

FIGS. 3A and 3B show placement of the gel 44 into the lacrimal canaliculus 18 using the lacrimal cannula 42 of the prior art. A distal tip of the lacrimal cannula 42 is inserted deep into the lacrimal canaliculus 18 to a location P1 (FIG. 3A), and the gel 44 is extruded from a distal tip of by depressing plunger 48 having a pusher engaging the gel from an initial position D1 to position D2 as the distal tip of the lacrimal cannula 42 is pulled back to position P2 (FIG. 3B) If the rate of injection of the gel is timed perfectly with the rate of withdrawal of the cannula, the canaliculus is completely filled without excess, as shown in FIGS. 4A to 4D.

FIGS. 5A and 5B, in contrast, show the result if the lacrimal cannula 42 of the prior art is withdrawn too slowly. The gel 44 will extrude back through the punctum 14 a since a tight seal cannot be maintained, and the volume of gel delivered into the canaliculus is uncontrolled. FIG. 5C shows the result if the lacrimal cannula 42 of the prior art is withdrawn too quickly. The lacrimal canaliculus 18 is only partially filled.

For these reasons, it would be desirable to provide improved lacrimal cannula and methods for accessing and filling a patient's lacrimal canaliculi. Such cannula and methods should both (1) improve sealing while delivering substances through the cannula to the lacrimal canaliculi and (2) reduce the risk of over insertion and potential damage to the lacrimal tissue. At least some of these objectives will be met by the inventions described hereinafter and in the claims.

2. Description of the Background Art. U.S. Pat. No. 5,593,393 describes a lacrimal cannula with multiple diameters. The cannula described in U.S. Pat. No. 5,593,393 is designed so that a small diameter tube is first placed into the punctum and as the cannula is advanced, the large diameter tube of the cannula penetrates the punctum into the canaliculus and seals the system. U.S. Pat. No. 3,540,447 discloses a spinal needle in which the pointed distal end has a smaller diameter than the proximal end. It can be seen, however, that the tip is sharp and not useful for lacrimal punctal. If used for lacrimal irrigation it could cause bleeding and irritation of the lacrimal tissues. This device was designed specifically for spinal injection and also is attached directly to a very specific syringe. Furthermore, this device would require extensive modification (changing the end point to an even point versus slanted, rounding and smoothing of the sharp tip, decrease in shaft length, and several changes in diameter) to be utilized for lacrimal insertion.

U.S. Pat. No. 3,216,616 discloses a reduced diameter point on a needle. This patent describes almost exclusively the improvements in syringe design and does not give specifics of the needle design. The device described would make lacrimal irrigation much more complex and would require modifications described in the patent above. U.S. Pat. No. 4,335,718 describes a needle cannula for penetration of the skin. Again, this device would require the extensive modification above to be utilized for lacrimal insertion.

### SUMMARY OF THE INVENTION

The present invention achieves at least some of these objectives by providing a lacrimal cannula having a distal tip which is sized and shaped to engage and seal against a punctal opening with minimum penetration into the lacrimal canalicula. Punctal dilation and irrigation can be performed a one-step process rather than the two-step process of the prior art. The present invention prevents the cannula from over-penetrating the lacrimal canaliculus, typically limiting insertion of a distal-most end of the cannula into the canaliculus so that it does not enter into a horizontal region of the canaliculus, typically limiting the insertion distance into a vertical region of the canaliculus to 2 mm or less. While lacrimal irrigation and gel delivery is still possible, the present invention prevents the distal-most end of the cannula from entering and probing deep into the lacrimal canalicula.

The distal tip of the lacrimal cannula is typically formed as a small-diameter, rounded end to allow controlled entry into the lacrimal puncta while minimizing further travel into the vertical region of the canaliculus. The small-diameter, rounded end transitions to larger diameter region which seals against a periphery of the puncta to both prevent or inhibit back flow of the gel or other material being introduced into the canalicular system and allow for complete filling of the canalicular system, including if necessary, the lacrimal sac. By providing a short length of the small diameter distal portion of the cannula, typically 1 mm to 2 mm, and a rapid increase in the diameter proximal to the small diameter portion, effective docking and sealing of the distal tip of cannula against the punctum is achieved. Effective docking anchors the distal tip in place and effective sealing and prevents backflow of the gel or other material being delivered.

In a first aspect, the present invention provides a lacrimal cannula which comprises a hollow tubular body. The hollow tubular body has a dispensing end, a loading end, and an injection lumen therebetween. The dispensing end of the hollow tubular body terminates in a distal tip which is configured to engage and seal against a patient's punctal os while being inhibited from entering a horizontal portion of a patient's lacrimal canaliculus.

In specific instances, the distal tip of the lacrimal cannula may have an outer diameter in a range from 0.3 mm to 0.7 mm, usually from 0.4 mm to 0.6 mm, at its distal-most end. Often, the distal tip will taper radially inwardly in a distal direction toward the distal-most end to facilitate entry into the punctal os. In such instances, where the distal tip is tapered, the tip will often have an outer diameter in a range from 0.7 mm to 2 mm, typically from 0.7 mm to 1 mm, at a first distance in a range from 1 mm to 3 mm in a proximal direction from the distal-most end. Thus, the distal tip may have a generally conical shape with the pointed or narrowest diameter at the distal-most end and a larger diameter at or near a proximal end thereof. Often, the conical surface will engage and seal against a periphery of the punctal os as the distal tip is being introduced into a vertical portion of the lacrimal canaliculus through the punctal os.

In specific instances, the distal tip may taper radially inwardly (i.e. converge) at a first angle in a range from 10° to 20° over the first distance. Such instances, the distal tip will often taper radially outwardly at a second angle greater than the first angle in a direction proximal of the first distance. For example, the second angle may be in a range from 25° to 60°, and the distal tip may diverge radially outwardly over a second distance in a range from 0.5 mm to 3 mm beyond the first distance.

The injection lumen may have a variety of configurations. For example, it may be tapered radially inwardly in a distal direction from the loading end to the dispensing end. Usually, the diameter of the injection lumen will be as large as possible at least within the distal portion of the hollow tubular body in order to reduce injection resistance. For example, the distal-most end of the distal tip of this dispensing end of the hollow tubular body will typically have a thin wall, often with a wall thickness between the outer surface of the body and an inner wall of the injection lumen in the range between 0.03 mm and 0.2 mm.

In other instances, the hollow tubular body of a lacrimal cannula according to the present invention comprises a distal-most section, a middle section, and a proximal section, wherein the distal most section is generally cylindrical having a length in a range from 1.5 mm to 3 mm and an outside diameter along its length in the range from 0.3 mm to 0.7 mm. The middle section typically has is generally cylindrical having a length in a range from 3 mm to 10 mm and an outside diameter along its length in the range from 3 mm to 10 mm, where the distal-most section may be straight and aligned with an axis of the hollow tubular body, may be straight and deflected at an angle in a range from 10° to 45° relative to an axis of the hollow tubular body, or may be curved.

The loading end of the hollow tubular body will typically be configured to be removably secured to a syringe, often having a luer fitting of a type selected to be coupled to a distal end of a syringe. This syringe will typically be pre-filled with a gel or other material which is to be delivered to the lacrimal canaliculus, and the present invention may further comprise syringe assemblies which include a lacrimal cannula in combination with a pre-loaded syringe.

In a second aspect, the present invention provides methods for delivering substance to a patient's lacrimal canaliculus. The methods comprise engaging a distal tip of a hollow tubular body against the patient's punctal os in a manner of such that the distal tip seals against the punctal os, but does not enter into the patient's horizontal lacrimal canaliculus. After the distal tip has been engaged and sealed against the punctal os, the substance is injected through an injection lumen within the hollow tubular body, out of the distal tip, and into the patient's lacrimal canaliculus. By injecting from a location at or shortly past the punctal os, the gel or other substance can be delivered in manner which fills the lacrimal canaliculus completely with no or minimum excess delivery.

In specific aspects of the methods of the present invention, a distal-most end of the distal tip may have a diameter at its distal-most end than can pass through the punctal os. In such instances, the distal tip usually tapers radially outwardly in a proximal direction so that an increased diameter of the distal tip will engage against a periphery of the punctal o as the distal tip is advance further into the vertical lacrimal canaliculus in order to block further advancement of the distal tip. In this way, the distal tip may extend partly into the patient's vertical lacrimal canaliculus, thus being prevented from entering the patient's horizontal lacrimal canaliculus. In further specific embodiments, the distal-most end of the distal tip will be blocked from entering the patient's vertical lacrimal canaliculus beyond a distance of 3 mm, often beyond a distance of 1 mm.

Further objects and advantages of this invention will become apparent from consideration of the drawings and ensuing description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings.

FIG. 1 illustrates the anatomy of the lacrimal system of the human eye.

FIGS. 2A and 2B illustrate a typical prior art lacrimal injector system including a dilator and a lacrimal cannula.

FIGS. 3A and 3B illustrate placement of a gel into the lacrimal canaliculus using the lacrimal cannula of FIGS. 2A and 2B.

FIGS. 4A to 4D provide a detailed view of a successful placement of a gel into the canaliculus using the cannula of FIGS. 2A and 2B.

FIGS. 5A and 5B show the result if the cannula of the prior art cannula of FIGS. 2A and 2B is withdrawn too slowly. FIG. 5C shows the result if the cannula of FIGS. 2A and 2B is withdrawn too quickly.

FIG. 6 is a cross-sectional view of a lacrimal injection of the present invention.

FIG. 6A is a detailed cross-sectional view taken along line 6A-6A of FIG. 6.

FIG. 7 illustrates the lacrimal cannula of FIGS. 6 and 6A mounted on a gel-filled syringe.

FIG. 8 shows the result of using the lacrimal cannula of FIGS. 6 and 6A to fill the canaliculus, and the lacrimal sac if necessary.

FIGS. 9A and 9B show the lacrimal cannula of FIGS. 6 and 6A mounted on a delivery bulb filled with gel.

FIGS. 10A and 10B show the lacrimal cannula of FIGS. 6 and 6A mounted on a gas drive filled with gel.

FIGS. 11A to 11D show a lacrimal cannula having an axially aligned straight stepped delivery tip (FIGS. 11A to 11C) delivering a gel into the canaliculus (FIG. 11D).

FIGS. 12A and 12B show a lacrimal cannula having an axially deflected straight stepped delivery tip (FIG. 12A) delivering a gel into the canaliculus (FIG. 12B).

FIGS. 13A and 13B show a lacrimal cannula having a curved stepped delivery tip (FIG. 13A) delivering a gel into the canaliculus (FIG. 13B).

FIGS. 14A to 14E illustrate the steps of using the lacrimal cannula of FIGS. 11A and 11B together with a conventional syringe to deliver a gel into the canaliculus of the eye of a patient.

### DETAILED DESCRIPTION OF THE INVENTION

The present invention allows for easy one step insertion (no punctal dilation is necessary) of materials into the lacrimal canaliculus and lacrimal sac. The larger diameter distal portion of the cannula allows for closure of the lacrimal puncta to avoid backwash of material during the insertion procedure, thus assuring that the lacrimal canaliculus and if necessary, the lacrimal sac are completely filled with material. The design prevents the device from going any further than about 2 mm into the canaliculus, thus preventing any displacement of material out of the canaliculus.

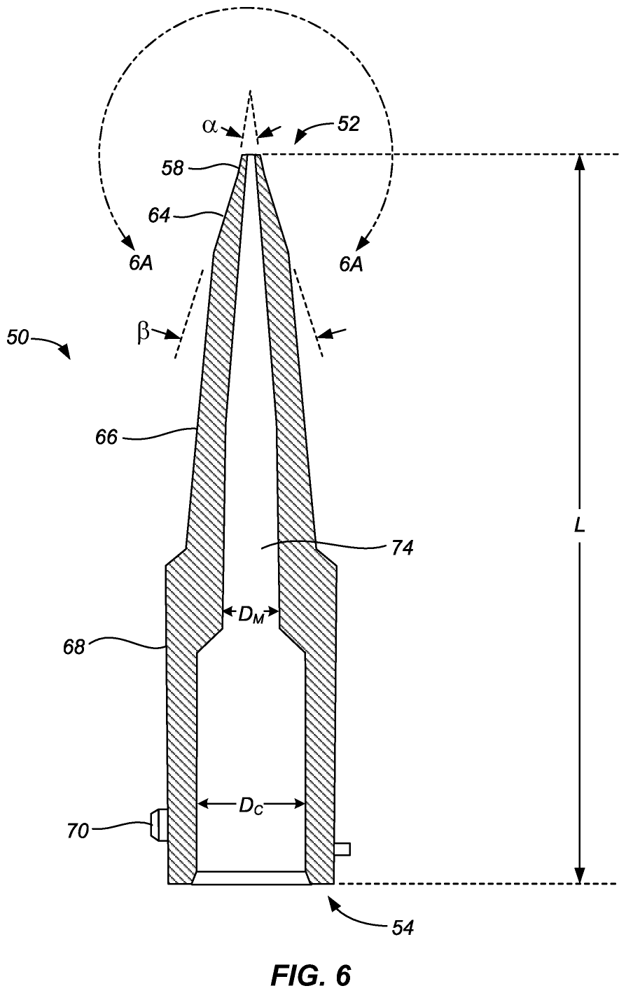

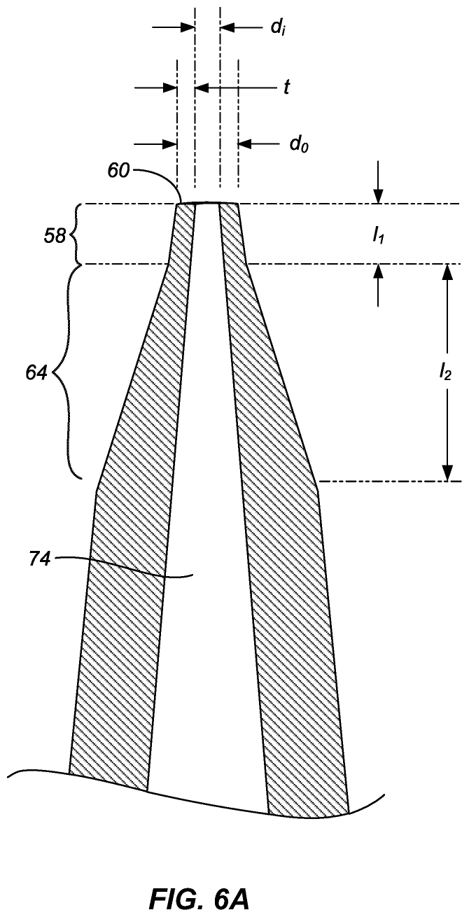

Referring now to FIGS. 6 and 6A, a lacrimal cannula 50 constructed in accordance with the principles of the present invention comprises a tubular body having a distal or dispensing end 52 and a proximal or loading end 54. An injection lumen 74 extends from the proximal end 54 to the distal end 52 to allow for the delivery of a gel or other substance from a syringe (as described herein below) out through a distal tip 58 of the cannula into a patient's punctal os.

The distal tip 58 of the lacrimal cannula 50 has an outer diameter do at its distal-most end. An opening report having an inner diameter di formed therein. The distal tip 58 will typically flare or taper radially outwardly in a proximal direction toward the proximal or loading end 54 over a first length I₁.

Immediately proximal of the distal tip 58, a tapered stop region 64 having a length I₂. The tapered stop region also flares or is tapered radially outwardly in the proximal direction toward the proximal or loading end 54. The distal tip 58 will typically diverge at an angle alpha a while the tapered stop region 64 will diverge radially outwardly an angle beta β. Angle β will be larger than angle α so that the tapered stop region will immediately halt distal advancement of the lacrimal cannula 50 into the punctal os if the advancement did not occur with the distal tip 58.

Proximal of the tapered stop region 64, a transition region 66 may be provided and proximal of the transition region a cylindrical shank region 68 may be formed. A luer or other attachment feature 70 will be provided at a proximal end of the cylindrical shank region 68 in order to permit attachment to a syringe, as will be described in more detail below.

Exemplary and preferred dimensions for the lacrimal cannula 50 of the present invention are set forth below in Table I.

TABLE I
DIMENSIONS

| DIMENSION | BROAD RANGE | NARROW RANGE | EXEMPLARY VALUE | |
|---|---|---|---|---|
| L | 15 mm-40 mm | 20 mm-30 mm | 25 | mm |
| l1 | 0.5 mm-5 mm | 0.5 mm-3 mm | 2 | mm |
| l2 | 2 mm-5 mm | 3 mm-4 mm | 3.4 | mm |
| α | 7.5°-25° | 10°-20° | 15° | |
| β | 20°-75° | 25°-60° | 40° | |
| dᵢ | 0.16 mm-0.6 mm | 0.16 mm-0.26 mm | 0.25 | mm |
| dₒ | 0.3 mm-0.7 mm | 0.4 mm-0.5 mm | 0.45 | mm |
| t | 0.03 mm-1 mm | 0.05 mm-0.5 mm | 0.1 | mm |
| D_{M} | 1 mm-4 mm | 2 mm-3 mm | 2.4 | mm |
| D_{C} | 3 mm-5 mm | 3.5 mm-4.5 mm | 4.2 | mm |

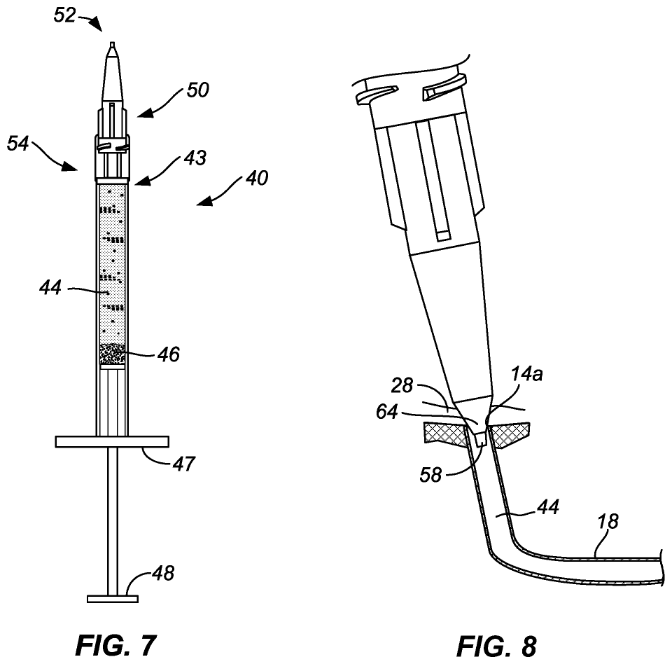

Referring now to FIG. 7, the lacrimal cannula 50 of the present invention may be attached to a syringe 47 similar to that described previously in connection with the prior art. The syringe 47 will have a luer or other attachment region 43 at its distal end which is capable of removably attaching to the luer or other fitting 70 on the lacrimal cannula. The syringe further includes a gel 44 to be delivered, a pusher 46, finger grips 47, and a plunger 48, all of which can be constructed similarly to the prior art syringe described above.

Referring now to FIG. 8, use of the combination syringe 40 and lacrimal cannula 50 for dispensing a gel 44 into the lacrimal canaliculus 18 is described. The distal tip 58 of the lacrimal cannula 50 is inserted against and into the punctal os 14. The tapered stop region 64 of the distal tip 58 “wedges” into the lacrimal os 14a so that a seal is formed about the periphery of the os and against a circumferential surface of the tapered stop region 64. After the seal is achieved, the plunger 48 may be depressed to extrude gel 44 through the distal tip 58 and into the lacrimal canaliculus 18, including both the vertical and horizontal regions of the lacrimal canaliculus. By avoiding need to pull back any portion of the cannula and/or to control the rate at which the cannula is drawn through the canaliculus, complete filling of the canaliculus lumen may be achieved with minimum or no excess gel being lost through the surface end of the lacrimal canaliculus.

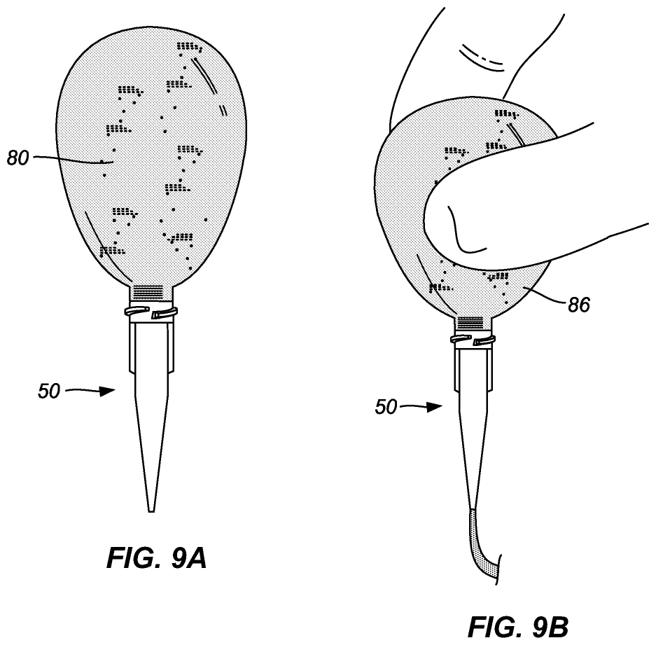

Referring now to FIGS. 9A and 9B, the particular dispensing tool used to inject gel or other material through the lacrimal cannula may be varied. For example, shown in FIGS. 9A and 9B, a bulb 80 may be filled with the gel and dispensed from the gel by squeezing to advance the gel material through the lumen 74 of the lacrimal cannula 50.

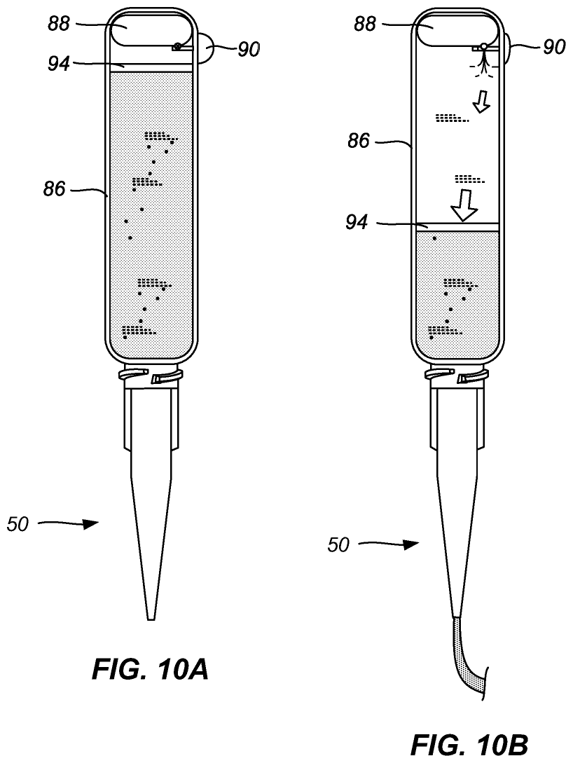

Alternatively, as shown in FIGS. 10A and 10B, a pressurized “gas drive” unit 88 may be attached to the proximal end of the lacrimal cannula 50 in order to dispense gel there through. For example, the gas driver 86 may include a pressurized cell or body 88 with a release mechanism 90, so that when it is desired to dispense the gel, the user may be able to depress the actuator 90 in order to release pressurized gas from the gas cylinder 88 so that piston 94 is moved forward to advance the gel through the cannula.

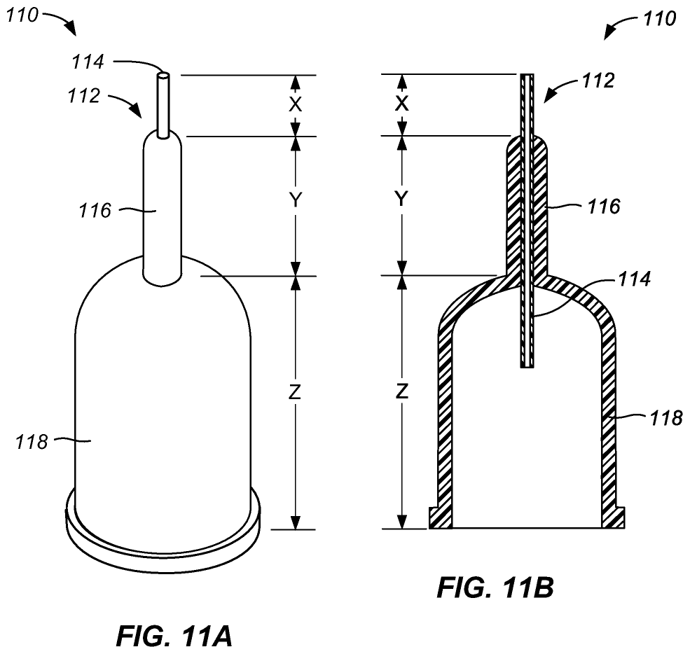

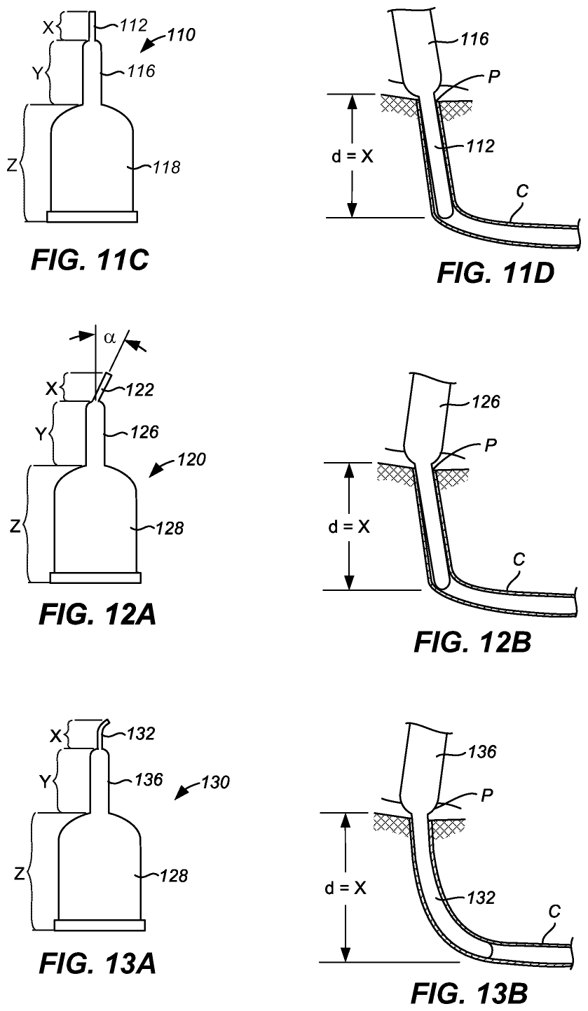

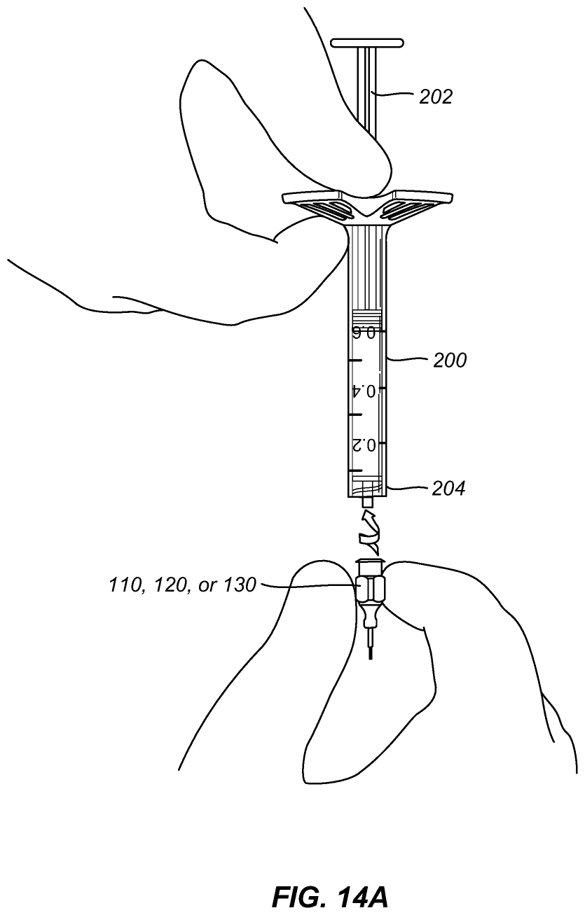

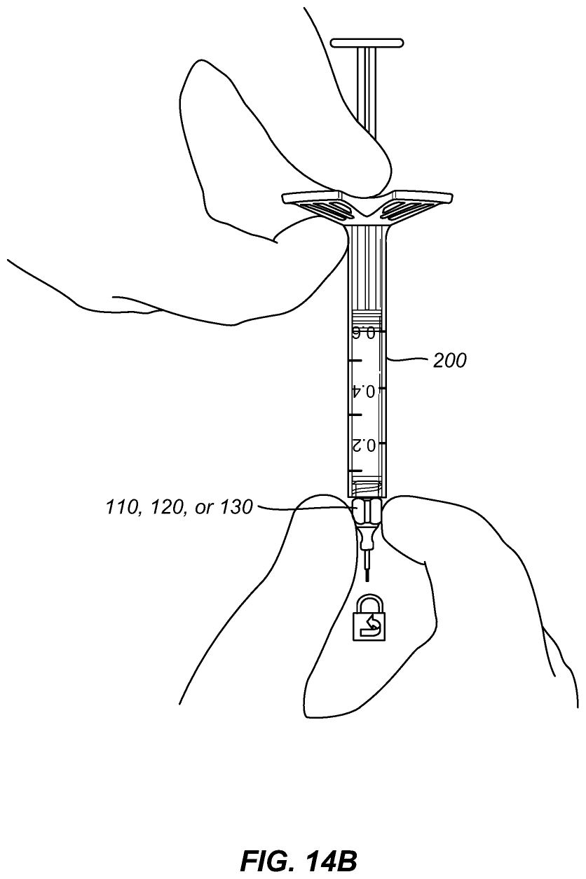

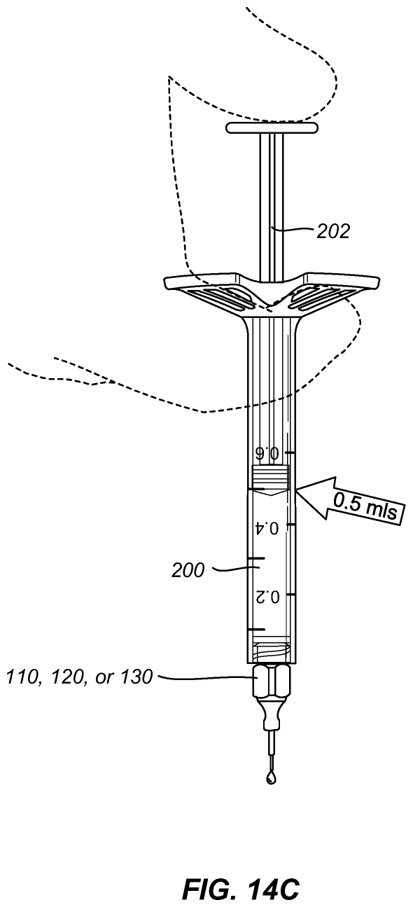

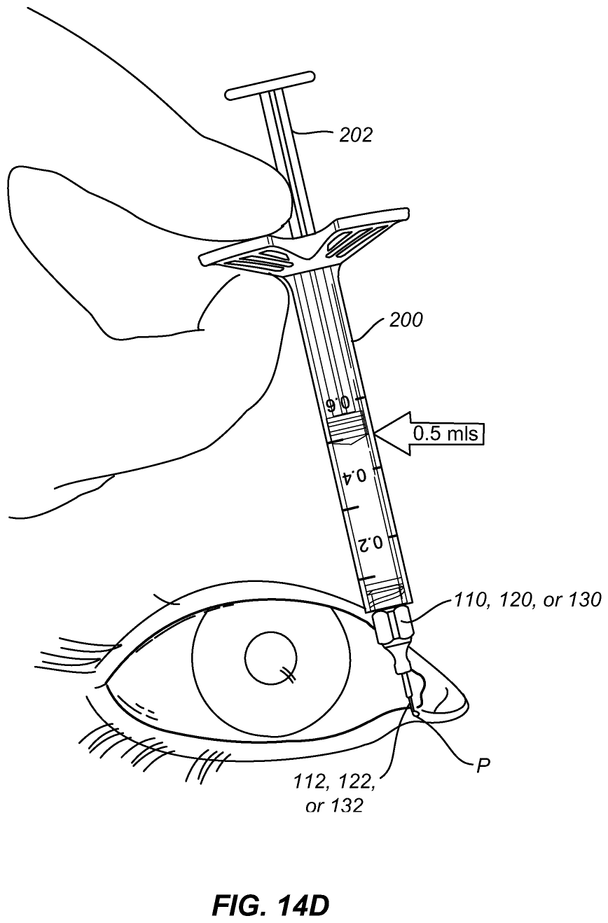

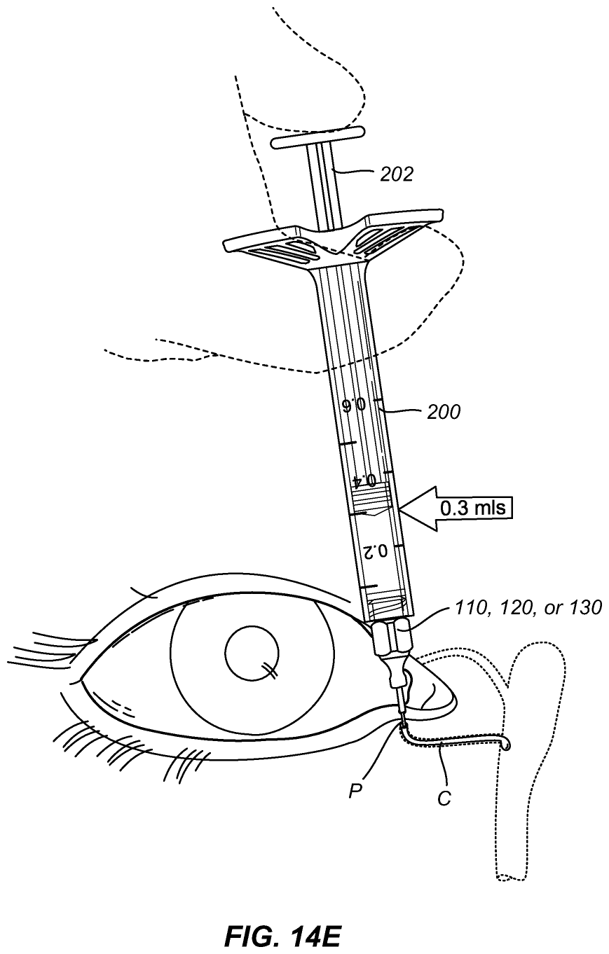

Referring now to FIGS. 11A to 11D, a disposable cannula 110 for delivery of materials into the canaliculus comprises a distal-most section 112 having a length X and configured to penetrate the patient's canaliculus to a precise depth X equal to the length X of the distal-most section 112. The distal-most section 112 is configured stop penetrating the punctum beyond the length X of the distal-most section 112. In a preferred aspect, the distal-most section 112 of the cannula 110 is configured to penetrate to a predetermined, proper depth without the use of a pre-placement tube as with the prior art. In a particularly preferred aspect, the distal-most section 112 may comprise a small diameter metal or other tube 114 which is sized to penetrate most, if not all, human puncta, without need for dilation, where penetration stops once a leading edge of the larger-diameter middle section 116, having a length Y reaches the opening of the punctum. A proximal section 116 of the cannula having a length Z is configured removably attach to a standard male Luer Lock on the end of a syringe 120 (FIGS. 14A to 14E) or other delivery device and has dimensions consistent with this function.

Distal-most section 112 comprises the small diameter tube 114 with dimensions selected to allow a distal portion of the tube which extends beyond a distal end of middle section 116 to be easily be inserted into the human punctum and remain in place without penetrating into the horizontal portion of the canaliculus. Middle section 116 has a larger diameter with dimensions selected to stop penetration of the cannula tip 110 once a distal leading edge or shoulder of the middle section engages with the punctum, at the same time not obscuring visibility for the subsequent delivery of material into the canaliculus. Exemplary and preferred dimension for the cannula tip 110 are set forth in Table II:

TABLE II
DIMENSIONS CANNULA 110

| DIMENSION | BROAD RANGE | NARROW RANGE | EXEMPLARY RANGE/VALUE | |
|---|---|---|---|---|
| Length of Distal- | 1.5 mm to 3 mm | 1.8 mm to 2.6 mm | 2 mm to 2.6 mm | |
| Most Section 112 (X) | | | | |
| O.D. of Distal-Most | 23 gauge to 30 gauge | 26 gauge to 28 gauge | 27 gauge | |
| Section 112 | (0.3 mm to 0.7 mm) | (0.35 mm to 057 mm) | (0.4 mm) | |
| I.D. of Distal-Most | 0.32 mm to 0.11 mm | 0.25 mm to 0.16 mm | 0.19 | mm |
| Section 112 | | | | |
| Length of Tube 114 | 4.5 mm to 15 mm | 6.8 mm to 13 mm | 11 | mm |
| Length of Middle | 3 mm to10 mm | 5 mm to7 mm | 6 | mm |
| Section 116(Y) | | | | |
| O.D. of Middle | >1.07 mm | >1.2 mm | 1.22 | mm. |
| Section 116 | | | | |
| Length of Proximal | 8 mm to 15 mm | 10 mm to 13 mm | 11 | mm |
| Most Section 118 (Z) | | | | |

While in some embodiments all portions of the disposable cannula 110 may be integrally formed, e.g. molded or machined, from a single block of material, it will often be preferred to form the small diameter tube 114 separately from the middle section 116. It will be particularly preferred to form the small diameter tube 114 from stainless steel or other medical grade metal while forming the middle section 116 and the proximal section 118 from a molded polymer. In some instances, the small diameter tube will have a length which is greater than the length of the middle section 116, allowing the manufacturer or user to adjustably position the small diameter tube 114 in the middle section to provide a variable length X, either at the time of manufacture or at the time of use.

As shown in FIG. 11D, the distal tip of the distal-most section 112 of the cannula tip 110 is inserted through the punctum P to a preselected depth d which is equal to the exposed length X of the distal-most section 112 to properly position the distal-most section within the canaliculus C.

As shown in FIGS. 12A and 12B, a cannula tip 120 is constructed similarly to the cannula tip 110 of FIGS. 11A to 11D except that a distal-most section 122 is angled relative to an axis of the cannula tip. An angle α between the axial direction and the direction of the distal-most section 122 will typically be in a range from 10° to 45°, usually from 15° to 35°.

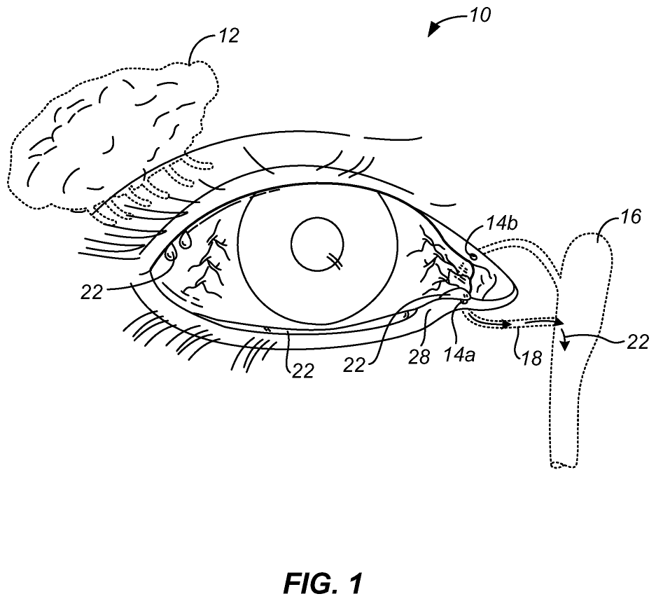

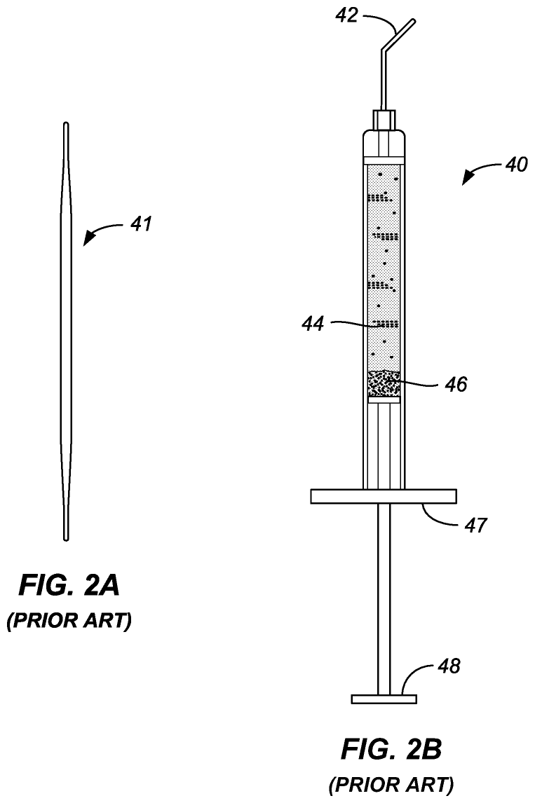

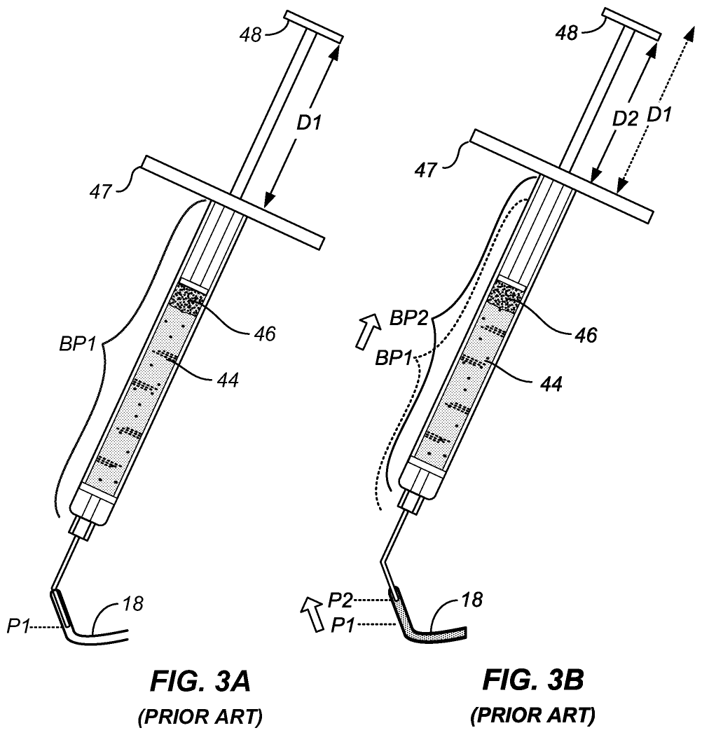

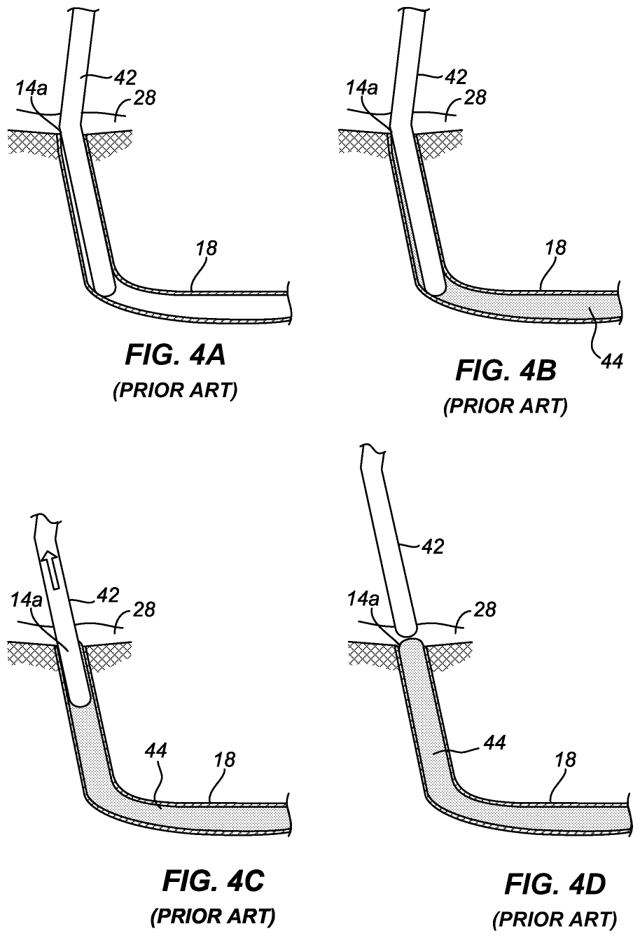

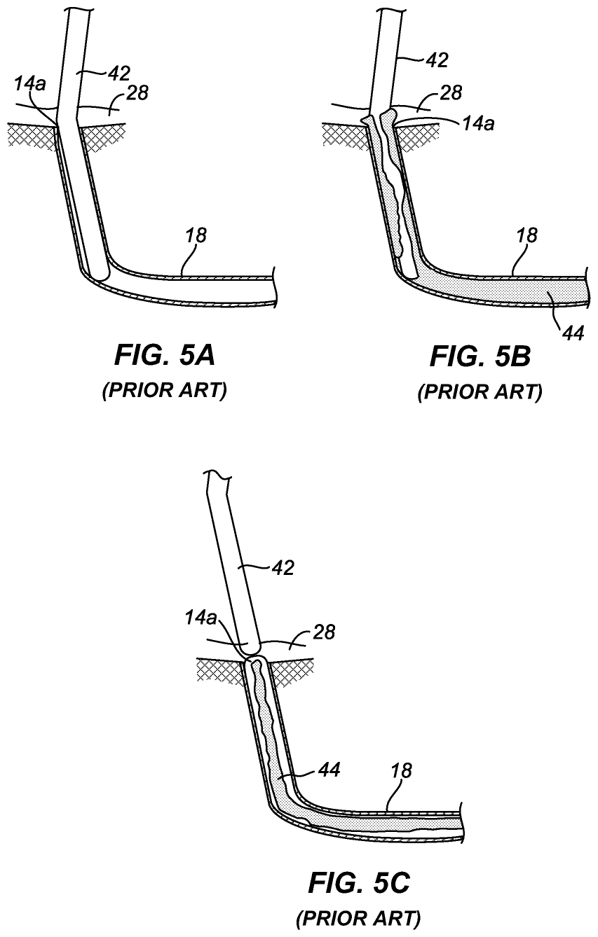

As shown in FIGS. 13A and 13B, a cannula tip 130 is constructed similarly to the cannula tip 110 of FIGS. 1A to 11D except that a distal-most section 132 curves away from an axis of the cannula tip.

Referring now to FIGS. 14A to 14E, any of the cannula tips of the present invention, such as cannula tips 110, 120, or 130, may be attached to a conventional syringe 200 having a plunger to 202 at one end and a luer connector 204 at the other end. The syringe will be filled with a gel, hydrogel or other material intended to be inserted into a patient's lacrimal canaliculus. The cannula tip 110, 120, or 130 is attached by simply screwing it into place as shown by the arrow in FIG. 14A. After locking the cannula tip in place, as shown in FIG. 14B, the user will depress the plunger 202 forward to extrude a small volume, generally about 0.1 ml of gel through the lacrimal cannula 110, 123, or 130. The volume of gel left in the syringe should be at least 0.5 ml. As shown in FIG. 14D, the distal tip of the distal-most section 112, 122, or 132 of the lacrimal cannula 110, 120, or 130 is advanced into the lower punctum P of the first eyelid, and 0.2 mL of the gel is extruded. Using the cannula tips of the present invention, it will rarely if ever be necessary to pre-dilate the patient's punctum P.

While the above description contains many specificities, these should not be construed as limitations on the scope of the invention. Many other variations are possible, for example:

1. The cannula may be made of metal, plastic, natural materials, or synthetic materials. The material of construction is preferably a material that can be injected molded, preferably a polyolefin, more preferably a polypropylene.

2. The outer diameter of the tip of the cannula will generally be less than or equal to about 0.6 mm, but this could vary. The inner diameter of the tip will generally be greater than or equal to 0.2, but this can vary depending on the properties of the material to be delivered.

3. The length of the small diameter portion of the cannula shaft is generally less than 2 mm, and the length of the large diameter portion can vary.

4. Taper length, taper angle, and taper positions could vary and still accomplish the same goal of insertion without the need for dilation of the punctum, docking (sealing against the punctum), prevention of entry of the cannula into the horizontal canaliculus, and delivery of material into the canaliculus and, if necessary, the lacrimal sac.

5. The hub connection is usually a Luer Lock, but it can vary depending on the device to which it is intended to be connected

6. Sidewall thickness could vary to allow for reinforcement of the cannula depending on the cannula material of construction.

7. The cannula will generally be manufactured as one connected piece, but could be made integrally or separately or in modular sections.

The present application is also related to methods for inserting implants into the nasolacrimal drainage system, with embodiments providing punctal and canalicular implants, and lacrimal sac implants with or without therapeutic agent delivery capabilities.

The present invention provides improved canalicular occlusion devices, systems and methods for insertion through the punctum and into the canaliculus of a patient. In many embodiments, the canalicular occlusion device can be reliably retained in the eye.

In a first aspect, embodiments of the present invention provide a canalicular occlusion device that can be inserted through the punctum and into the canaliculus of a patient using the delivery cannula described above. Specific and known volumes of gel can be delivered since the cannula of the present invention prevents backflow of the gel and the gel can only advance forward into the canaliculus and, if desired, the lacrimal sac. The canalicular occlusion device may comprises a gel which may or may not contain a therapeutic agent.

In many embodiments, the gel is a hydrogel. The gel may be particulated so that it can be easily injected through the cannula of the present invention and into the punctum and canaliculus, and the lacrimal sac if desired. The gel may comprise hyaluronic acid. The hyaluronic acid may be crosslinked.

In preferred embodiments, the canalicular occlusion device may inhibit tear fluid flow through the canalicular lumen and lacrimal sac if desired.

In many embodiments, a canalicular occlusion device for insertion through a punctum of a patient and into the canaliculus and lacrimal sac, if desired, is provided.

In many embodiments, a canalicular occlusion device for treating an eye is provided. The eye has a tear fluid, a punctum, a canaliculus, and a lacrimal sac.

In specific embodiments, the canalicular occlusion device is capable of resilient expansion to totally occlude the canaliculus of the eye.

It is understood that the present invention is not limited to the particular construction and arrangement disclosed, but embraces all such modified forms. Its scope is to be determined by the appended claims rather that by examples given.

What is claimed is:

1. A lacrimal cannula comprising:
a hollow tubular body having a dispensing end, a loading end, and an injection lumen theretween;
wherein the dispensing end terminates in a distal tip configured to engage and seal against a punctal os while being inhibited from entering a horizontal portion the lacrimal canaliculus.

2. A lacrimal cannula as in claim 1, wherein the distal tip has an outer diameter in a range from 0.3 mm to 0.7 mm at its distal-most end.

3. A lacrimal cannula as in claim 2, wherein the distal tip tapers radially inwardly in a distal direction toward the distal-most end.

4. A lacrimal cannula as in claim 3, wherein the distal tip has an outer diameter in a range from 0.7 mm to 2 mm at a first distance in a range from 1 mm to 3 mm from the distal-most end.

5. A lacrimal cannula as in claim 4, wherein the distal tip tapers radially inwardly at a first angle in a range from 10° to 20° over the first distance.

6. A lacrimal cannula as in claim 5, wherein the distal tip tapers radially outwardly at a second angle in a range from 25° to 60° over a second distance in a range from 2 mm to 5 mm from the distal-most end.

7. A lacrimal cannula as in claim 6, wherein the injection lumen is tapered radially inwardly in a distal direction from the loading end to the dispensing end.

8. A lacrimal cannula as in claim 7, wherein the distal-most end of the distal tip of the dispensing end of the hollow tubular body has a wall thickness between the outer surface and an inner wall of the injection lumen in a range between 0.03 mm and 0.2 mm.

9. A lacrimal cannula as in claim 8, wherein the loading end of the hollow tubular body is configured to be removably secured to a syringe.

10. A lacrimal cannula as in claim 2, wherein the hollow tubular body comprises a distal-most section, a middle section, and a proximal section, wherein the distal most section is generally cylindrical having a length in a range from 1.5 mm to 3 mm and an outside diameter along its length in the range from 0.3 mm to 0.7 mm.

11. A lacrimal cannula as in claim 10, wherein the middle section is generally cylindrical having a length in a range from 3 mm to 10 mm and an outside diameter along its length in the range from 3 mm to 10 mm.

12. A lacrimal cannula as in claim 10, wherein the distal-most section is straight and aligned with an axis of the hollow tubular body.

13. A lacrimal cannula as in claim 10, wherein the distal-most section is straight and deflected at an angle in a range from 10° to 45° relative to an axis of the hollow tubular body.

14. A lacrimal cannula as in claim 10, wherein the distal most section is curved.

15. A syringe assembly comprising:
a lacrimal cannula as in any one of claims 1 to 10; and
a syringe.

16. The syringe assembly of claim 15, wherein the syringe is pre-loaded with a substance to be delivered to a patient's lacrimal canaliculus.

17. A method for delivering a substance to a patient's lacrimal canaliculus or lacrimal sac, said method comprising:
engaging a distal tip of a hollow tubular body against the patient's punctal os, wherein the distal tip seals against the punctal os but does not enter the patient's horizontal lacrimal canaliculus; and
injecting the substance through an injection lumen of the hollow tubular body, out of the distal tip, and into the patient's lacrimal sac and/or lacrimal canaliculus.

18. A method as in claim 17, wherein a distal-most end on the distal tip has a diameter that can fit within the punctal os.

19. A method as in claim 18, wherein the distal tip tapers radially inwardly in a distal direction toward the distal-most end so that an increased diameter of the distal tip at a location proximal of the distal-most end will block advancement of the distal tip into the patient's vertical lacrimal canaliculus.

20. A method as in claim 19, wherein the distal-most end of the distal tip is blocked from entering the patient's vertical lacrimal canaliculus beyond a distance of 3 mm.

21. A method as in claim 20, wherein the distal-most end of the distal tip is blocked from entering the patient's vertical lacrimal canaliculus beyond a distance of 1 mm.

22. A method as in claim 17, wherein the substance comprises a gel.
